(19) United States
(12) Patent Application Publication
(10) Pub. No.: US 2007/0248555 A1
(43) Pub. Date: Oct. 25, 2007

(54) Composition and method for managing redness of skin associated with dermatological condition

(76) Inventor: Dana M. Watson, Raleigh, NC (US)

Correspondence Address: H. JAY SPIEGEL - H. JAY SPIEGEL & ASSOCIATES, P.O. BOX 11, MOUNT VERNON, VA, 22121, US

(21) Appl. No.: 11/407,239

(22) Filed: Apr. 20, 2006

(51) Int. Cl. A61K 8/81; A61K 8/73; A61K 8/37

(52) U.S. Cl. 424/70.13; 424/70.16; 424/70.31

(57) ABSTRACT

The present invention is directed towards a topical composition for managing redness of skin associated with a dermatological condition, comprising water, gelled ethyl alcohol (an anti-bacterial), anti-oxidants, thickening agents, emulsifiers, stabilizers, and a natural esterified anti-inflammatory (Hepes). This combined therapy is more effective than anti-bacterial agents alone and is particularly effective for those who do not respond well to the solutions containing only anti-bacterial agents.

### BACKGROUND OF INVENTION

The present invention relates to the treatment of a dermatological condition and, more specifically, to the treatment of rosacea, by a topical application of a composition comprising a combination of gelled ethyl alcohol and Hepes Linoleate. The invention relates to topical compositions for the treatment of a dermatological condition and, more specifically, to the treatment of rosacea. The method for treatment preferably includes two to three applications daily to the affected area of the skin.

Rosacea is a chronic inflammatory skin disorder that can develop in the third to sixth decade of life characterized by four distinct clinical stages predominately affecting the central aspect of the face. The disease appears to be more prevalent in northern climates where cold exposure is experienced more often, and in light-skinned persons on whom flushing is common and sensitivity to sunlight is particularly high. The first clinical evidence of rosacea is frequent and causes intense vasodilation or flushing. Most patients progress to a vascular stage characterized by display of an erythema that can persist for hours or days after a triggering event. Some patients remain stabilized at this stage while some progress to an inflammatory stage characterized by display of an array of papules and pustules in addition to the persistent erytherma. This stage can become a chronic condition. A few patients, mostly male, can progress to the final stage characterized by a distinctive hyperplasia or swelling, especially of the nose. Rosacea is a visible skin condition that can have a high impact on the quality of life of the patient.

Successful management of rosacea begins with early diagnosis and treatment. Treatment is generally aimed at controlling the symptoms and making the skin look better. Currently there is no cure for rosacea, though the frequency of its flare-ups can be diminished and their severity reduced. Most cases of rosacea can be controlled with anti-inflammatory medications combined with avoiding lifestyle and environmental factors that may aggravate the disorder in individual cases. Treatment generally works best at improving the papules and pustules of rosacea; the redness of the skin is more difficult to treat. There are two groups of therapeutic agents for inflammatory rosacea conditions: (1) systemic and topical antibiotics; and (2) retinoids. Systemic and topical antibiotics include tetracycline, metronidazole, erythromycin, minocycline, and clindamycin, but the use of these agents is often accompanied by drug side effects, the development of resistance, and changes in the normal microbial flora. Retinoids include treinoin (vitamin A or retinoic acid), which is applied topically to inhibit follicular keratinization, and isotretinoin (13-cis-retinoic acid), which is administered systematically to suppress the activity of the sebaceous glands. Retinoids are often irritants and are not advised for individuals with sensitive skin. Retinoids can also be phototoxic and they can induce thin and easily bruisable, fragile skin.

Metronidazole (5-methyl-5nitroimidazole-1-ethanol), and antibacterial, is currently one of the more frequently prescribed treatments for rosacea in the United States. It is available as a topical cream known as Metrogel.™. from Galderma. Metronidazole is structurally similar to some materials which are believed to be carcinogens and is, in fact, listed by the U.S. Environmental Protection Agency as reasonably anticipated to be a human carcinogen. See Merck Index, 1996, page 1051.

U.S. Pat. No. 5,972,993, Ptchelintsev, issued Oct. 26, 1999, describes a method for treating rosacea and sensitive skin conditions using certain specifically-defined antioxidants.

U.S. Pat. No. 5,952,372, McDaniel, issued Sep. 14, 1999, describes a method for treating rosacea using oral or topically applied ivermectin. This method of treatment is aimed at reducing or eliminating the Demodex organisms which are frequently found on the skin of rosacea patients.

U.S. Pat. No. 5,998,395, Klingman, issued Dec. 7, 1999, describes a method for suppressing inflammation in an inflammatory dermatosis, such as rosacea, using a topically applied composition containing both a corticosteroid and a retinoid.

U.S. Pat. No. 6,028,118, Dupont et al., Feb. 22, 2000, describes the use of shark cartilage extract as an anti-angiogenic, anti-inflammatory and anti-collagenolytic material which may be used in the treatment of rosacea.

U.S. Pat. No. 5,994,330, El Khoury, issued Nov. 30, 1999, describes the treatment of acne and other inflammatory skin conditions using topically administered muscarinic agents. The therapeutic effects of the invention are said to include a decrease in redness, swelling and inflammation.

U.S. Pat. No. 5,968,532, De Lacharriere, et al., issued Oct. 19, 1999, describes the use of an ethylene diamine derivative in a cosmetic or dermatological composition containing a material having an irritant side effect. The ethylene diamine derivative is said to minimize skin irritation, erythema and sensations of inflammation or rosacea stemming from the use of the cosmetic/dermatological product.

U.S. Pat. No., 5,885,595, Corey, et al., issued Mar. 23, 1999, describes a cosmetic composition which includes a retinal fatty acid ester. The composition is said to be effective for treating chronoaging conditions of the skin and dermatological disorders including acne, follicular and lesional papules, actinic kertoses, oily skin and rosacea.

U.S. Pat. No. 6,071,541, Murad, issued Jun. 6, 2000, describes the use of a topical composition which contains a hydroxy acid or tannic acid to exfoliate a portion of the skin, stabilized hydrogen peroxide to facilitate cleansing of the skin, and an antimicrobial agent to inhibit or reduce microorganisms of the skin. The composition is said to be effective in the treatment and management of inflammatory skin conditions, such as acne and acneiform rosacea.

U.S. Pat. No. 6,057,341, Charpentier, issued May 2, 2000, describes pharmaceutical or cosmetic composition which include novel bi-aromatic dibenzofuran derivatives. The compositions are said to exhibit pharmacological responses of the retinoid agonist type and are further said t be effective in treating keratinization disorders, including acne rosacea.

U.S. Pat. No. 5,886,233, Steinmeyer, et al., issued Mar. 23, 1999, describes cyclohexanone derivatives used to synthesize vitamin D compounds. The compounds are said to be useful for treating skin, such as in the treatment of acne.

There remains a need in the art for a more effective and otherwise improved methods for treating dermatological conditions related to rosacea by, for example, topically applying compositions, having a desired degree of effectivity, to areas of the skin of a patient in need thereof.

### SUMMARY OF INVENTION

According to the present invention, the chronic inflammatory skin disorder, rosacea, can be controlled and cleared more effectively than with the use of topical compositions in the prior art by administering to the affected areas of the skin a composition comprising water, gelled ethyl alcohol, thickening agent, emulsifiers, moisturizers, stabilizers and a new chemical entity that blocks the inflammatory cascade at the level of phospholipase A₂ (Hepes Linoleate). Specifically, the gelled ethyl alcohol and the Hepes compound have a synergistic effect when in a single formulation which leads to a more rapid clearing and is notably effective in the treatment of rosacea and reducing the redness in the skin. Typically rosacea can be controlled and cleared by once or twice daily applications of a composition comprising water, gelled ethyl alcohol, thickening agent, emulsifiers, moisturizers, stabilizers and an anti-inflammatory (Hepes Linoleate).

Thereafter, clearance can be maintained by less frequent and or less potent applications of the composition.

### DETAILED DESCRIPTION OF THE PREFERRED EMBODIMENTS

As noted above, in one embodiment, disclosed is a method for treating rosacea of the skin of a patient by topically applying to an area of the skin in need thereof, an effective amount of a composition comprising gelled ethyl alcohol combined with the Hepes compound. As used herein the word “treat,” “treating” or “treatment” refers to using the compositions of the present invention therapeutically to ameliorate an existing condition characterized by rosacea inflammatory skin conditions, and acne vulgaris.

The present invention is based also in part on the use of a non-steroidal, anti-inflammatory agent. This composition comprising water, SD39C Alcohol, thickening agent, emulsifiers, stabilizers, and an anti-inflammatory (Hepes linoleate) has a significant effect on the reduction of swelling, relieving sensitivity and redness associated with inflammatory acne conditions. Any inflammation that occurs in the body is the end result of a series of events known as the arachidonic acid cascade. The process starts with a traumatic or chemical event that leads to injury of the cell membrane. Cell membranes consist of phosolipids, which are complex lipid materials that contain fatty acids, one of which is arachidonic acid. In the initiation of the inflammatory process the first step is the conversion of arachidonic acid into the specific mediators of inflammation, which follows two pathways. One of the pathways is called the cyclooxygenase pathway and the other is called the lipoxygenase pathway. All anti-inflammatory agents block the formation of the end products of these two pathways. Aspirin for example blocks the cyclooxygenase pathway, while cortisone block both pathways by interfering with the formation of arachidonic acid. HO-1(Hepes linoleate) works in the same fashion, blocking both pathways and interfering with the formation of arachidonic acid and is the only non-steroidal compound known for blocking both of these pathways. (Pugliese, et al., September 2000)

The topical compositions of the present invention include water, an alcohol, an alpha hydroxy acid, a moisturizer, an isosorbide and a detergent. The amount of water present in the compositions of this invention may be from about 30 weight percent to about 70 weight percent, based upon the weight of the composition.

Preferably, the amount of water present is from about 35 weight percent to about 70 weight percent.

Organic peroxides which may be included in the topical compositions of the present invention could include any pharmaceutically acceptable organic peroxide, such as, for example, benzoyl peroxide, lauroyl peroxide, and carbamide peroxide. Preferably, the organic peroxide is benzoyl peroxide. The amount of organic peroxide present in the compositions of the invention may be from about 1 weight percent to about 20 weight percent, based upon the weight of the composition. Preferably, the organic peroxide is present in an amount from about 2.5 weight percent to about 10 weight percent.

Alpha hydroxy acids which may be included in the topical compositions of the present invention include any pharmaceutically acceptable alpha hydroxy acid, such as, for example, glycolic acid, lactic acid, 2-hydroxydecanoic acid, 2-hydroxystearic acid and malic acid. Preferably, the alpha hydroxy acid is one that is commonly used in topical compositions for treating acne, such as glycolic acid or lactic acid. Most preferably, the alpha hydroxy acid is glycolic acid. The amount of alpha hydroxy acid present in the compositions of the invention may be from about 0.1 weight percent to about 15 weight percent, based upon the weight of the composition. Preferably, the alpha hydroxy acid is present in an amount from about 1 weight percent to about 10 weight percent.

Moisturizers which may be included in the topical compositions of the present invention include any pharmaceutically acceptable moisturizer, such as, for example, sodium pyrollidone carboxylate, glycerin, glycolic acid, propylene glycol and sorbitol. Preferably, the moisturizer is sodium pyrollidone carboxylate. The amount of moisturizer present in the compositions of the invention may be from about 0.5 weight percent to about 20 weight percent, based upon the weight of the composition. Preferably, the moisturizer is present in an amount from about 1 weight percent to about 10 weight percent.

Isosorbides which may be included in the topical compositions of the present invention include any pharmaceutically acceptable isosorbide. Such isosorbides include, for example, dimethyl isosorbide, diethyl isosorbide, and ethylmethyl isosorbide. Preferably, the isosorbide is an alkyl ester of isosorbide, such as dimethyl isosorbide. The amount of isosorbide present in the compositions of the invention may be from about 0.05 weight percent to about 20 weight percent, based upon the weight of the composition. Preferably, the isosorbide is present in an amount from about 0.05 weight percent to about 10 weight percent.

Detergents which may be included in the topical compositions of the present invention include any pharmaceutically acceptable detergent. Such detergents include, for example, sodium potassium lauryl sulfate, cocamidopropyl betaine, sodium cocoylisethionate, and disodium cocoamphopropionate. Preferably, the detergent is sodium potassium lauryl sulfate or cocamidopropyl betaine. The amount of detergent present in the compositions of the invention may be from about 15 weight percent to about 60 weight percent, based upon the weight of the composition. Preferably, the detergent is present in an amount from about 25 weight percent to about 40 weight percent.

The compositions of the present invention also may contain various other ingredients that are commonly included in topical pharmaceutical compositions. Such ingredients include, for example, thickeners, preservatives, binders, wetting agents, and bases.

Thickeners which may be included in the topical compositions of the present invention include any pharmaceutically acceptable thickener. Such thickeners include, for example, cetostearyl alcohol, corn starch, polyethylene glycol, PEG-14M (PEG-14M is available from Amerchol Corp., Edison, N.J.), xanthan gum, cetyl alcohol, bentonite, carbomer, PEG 12, and magnesium aluminum silicate. The thickeners may be present in the compositions of the invention in an amount from about 1 weight percent to about 30 weight percent, based upon the weight of the composition. Preferably, the thickener is present in an amount from about 2 weight percent to about 25 weight percent.

Preservatives which may be included in the topical compositions of the present invention include any pharmaceutically acceptable preservative. Such preservatives include, for example, methylparaben, propylparaben, imidurea, Potassium Sorbate, Phenacetin, and quatemium-15. Preferably, the preservative is methylparaben or imidurea. The amount of preservatives present in the compositions of the invention may be from about 0.05 weight percent to about 1 weight percent, based upon the weight of the composition. Preferably, the preservatives are present in an amount from about 0.1 weight percent to about 0.7 weight percent.

Bases which may be included in the topical compositions of the present invention include any pharmaceutically acceptable base. Such bases include, for example, sodium hydroxide, sodium citrate, sodium acetate, sodium phosphate, and sodium lactate.

Emollients which may be included in the topical compositions of the present invention include any pharmaceutically acceptable emollient. Such emollients include Glyceryl Stearate SE, Glyceryl Stearate, Isopropyl Myrisate, and Aluminium Hydroxide. Preferably the emollients are present in and amount from about 0.1 weight percent to 0.7 weight percent.

The general therapeutic regimen or strategy using the combination of the present invention preferably involves once to twice daily applications, preferably twice daily, of the combination to bring the acne under control. Thereafter, depending on the characteristics of the condition it is possible to maintain clearance by judicious application of the composition less frequently and/or in lower concentrations.

Controlled studies on a significant number of patients have shown the combined therapy according to the present invention is not only additive, but may truly be synergistic. That is, the combination is more effective than and produces responses not obtained by the usual treatment regimens of a corticosteroid, metronidazole, sulfacetamide, sulfur, and azelaic acid therapies alone. Thus, Rosacea, the common chronic skin condition characterized by a spectrum of clinical indications including flushing episodes, erythema, telegiectasia, inflammatory papulapustular eruptions resembling acne, and ocular symptoms clear more rapidly with the combination. Moreover, relapses are delayed and less severe. Significantly, improvement has been demonstrated in conditions which have become refractory to standard of conventional treatments.

It is important to note that Hepes is a compound that has a taurine molecule as one of the major components. Taurine is an important amino acid in the regulation of neurotransmitters, an immune function and in the control of inflammation. Its physiological mechanism is quite complex, but is known to inhibit the lipoxygenase pathway in the arachidonic acid cascade. The target leukotriene has been reported to be LTB4. Another reaction is the regulation of intracellular calcium and the inhibition of protein kinase C. These actions suggest that hepes is a significant agent to reduce cellular inflammation and cellular proliferation. (Pugliese, et al., September 2000) The key to this invention is the use of a new molecule, a hepes ester, which makes a whole new entity. All other applications in the literature that describe hepes as an anti-inflammatory agent use a delivery system that is by injection of venous infusion. Topical Hepes, Ehtane Sulphonic Acid, (not Hepes Oleate) has been used topically for arthritis and Rheumatis, as well as some applications for psoriasis, O'Sullivan U.S. Pat. Nos. 4,544,656 and 4,753,942. These applications did not include the ester.

In one application, a method of treating psoriasis and similar related ailments comprised applying to an affected skin area a therapeutically effective amount of at least one skin-compatible zwitteronic aminosulfonic acid of the formula wherein R is a straight or branched chain aliphatic radical, or RN is substituted or unsubstituted nitrogen-containing heterocycle which may have one additional hetero atom; and R′ is a C.sub2-4 straight or branched chain alkylene radical, O'Sullivan U.S. Pat. No. 4,544,656. In the second application, a method of treating arthritis and rheumatism in human patients comprises the topical application to an affected part of the patient's body of at least one zwitteronic aminosulphonic acid. The acid is made up as a pharmaceutical composition such as a cream and applied at a dosage of 50 mu.g-50 mg of the acid per day for at least 5 days, O'Sullivan U.S. Pat. No.4,753,942. It is notable that although these applications are referring to skin conditions and inflammatory conditions that neither of these inventions have incorporated the esterified ester chain that is associated with the hepes Oleate or Hepes Linoleate as used in the present invention.

The present invention will now be described in more detail with reference to the following specific, non-limiting examples. In these cases the composition was extensively evaluated and unless otherwise indicated the combination brought about rapid resolution (control and clearing) within two-three days of twice daily applications. The preferred ranges for the constituent ingredients are as follows:

| | |
|---|---|
| SD39C Alcohol | 2-60% |
| Aloe Vera | .1-10% |
| Caprylic/Capric Triglyceride | .1-10% |
| Hepes Linoleate | .5-20% |
| Evening Primrose Oil | .1-15% |
| Carbopol 981 | .12-1% |
| Sodium Ascorbyl Phosphate | .1-15% |
| Sorbitan Oleate | .1-10% |
| Tetrasodium EDTA | .05-10% |
| Acrylates/C10-30 Alkyl Acrylate Crosspolymer | .1-10% |
| Vitamin D3 Cholecalcipherol | .1-15% |
| Peppermint Oil USP | .1-10% |
| Balance Deionized Water | |
| | Total 100% Composition |

### EXAMPLE 1

Using the Listed Concentrations of the Present Invention:

| | |
|---|---|
| Deionized Water | 48.60% |
| SD39C Alcohol | 35.00% |
| Aloe Vera | 3.00% |
| Caprylic/Capric Triglyceride | 5.00% |
| Hepes Linoleate | 4.00% |
| Evening Primrose Oil | 2.00% |
| Carbopol 981 | 1.00% |
| Sodium Ascorbyl Phosphate | 0.50% |
| Sorbitan Oleate | 0.30% |
| Tetrasodium EDTA | 0.20% |
| Acrylates/C10-30 Alkyl Acrylate Crosspolymer | 0.20% |
| Vitamin D3 Cholecalcipherol | 0.10% |
| Peppermint Oil USP | 0.10% |

Approximately 10 cases of dermatological conditions related to rosacea were treated with the composition. In approximately 85% of the group, the condition was brought under control within two to three weeks of twice daily applications.

### EXAMPLE 2

Using the Listed Concentrations of the Present Invention:

| | |
|---|---|
| Deionized Water | 64.60% |
| SD39C Alcohol | 10.00% |
| Aloe Vera | 10.00% |
| Caprylic/Capric Triglyceride | 5.00% |
| Hepes Linoleate | 6.00% |
| Evening Primrose Oil | 2.00% |
| Carbopol 981 | 1.00% |
| Sodium Ascorbyl Phosphate | 0.50% |
| Sorbitan Oleate | 0.30% |
| Tetrasodium EDTA | 0.20% |
| Acrylates/C10-30 Alkyl Acrylate Crosspolymer | 0.20% |
| Vitamin D3 Cholecalcipherol | 0.10% |
| Peppermint Oil USP | 0.10% |

Approximately 15 cases of dermatological conditions related to rosacea were treated with the composition. In approximately 70% of the group, the condition was brought under control within two to three weeks of twice daily applications.

### EXAMPLE 3

| | |
|---|---|
| Deionized Water | 58.60% |
| SD39C Alcohol | 25.00% |
| Aloe Vera | 5.00% |
| Caprylic/Capric Triglyceride | 5.00% |
| Hepes Linoleate | 2.00% |
| Evening Primrose Oil | 2.00% |
| Carbopol 981 | 1.00% |
| Sodium Ascorbyl Phosphate | 0.50% |
| Sorbitan Oleate | 0.30% |
| Tetrasodium EDTA | 0.20% |
| Acrylates/C10-30 Alkyl Acrylate Crosspolymer | 0.20% |
| Vitamin D3 Cholecalcipherol | 0.10% |
| Peppermint Oil USP | 0.10% |

Approximately 20 cases of dermatological conditions related to rosacea were treated with the composition. In approximately 90% of the group, the condition was brought under control within two to three weeks of twice daily applications.

In these examples, the patients chosen ranged in ages from 25 to 55 years old and ranged in skin tones form very light to medium complexions.

1. A topical composition for the managing of redness in skin associated with dermatological conditions consisting essentially of:
a. Deionized Water present in an amount from about 25% to 70% by weight of the composition;
b. SD39C Alcohol present in an amount from 2% to 60% by weight of the composition;
c. Hepes Linoleate present in an amount from about 0.5% to 20% by weight of the composition;
d. A moisturizer selected from the group consisting of sodium pyrollidone carboxylate, glycerin, propylene glycol, sorbitol, Caprylic/Capric Triglyceride, Aloe Vera gel and mixtures thereof present in an amount from about 0.1% to 10% by weight of the composition;
e. An emulsifier selected from the group consisting of PEG-12, Glyceryl Stearate SE, Glyceryl Stearate, Isopropyl Myristate, Aluminum Hydroxide, xanthum gum, Carbopol 981 and mixtures thereof and present in an amount from about 0.1% to 20.0% by weight of the composition;
f. Thickeners selected from the group consisting of Bentonite, Cetyl Alcohol, Carbomer, Sorbitan Oleate, Acrylates/C10-30 Alkyl Acrylate Crosspolymer, and mixtures thereof and present in an amount from about 0.1% to 10.0% by weight of the composition;
g. Anti-oxidants selected from the group consisting of Vitamin A (Retinyl Palmitate), Vitamin Tri-C (Ascorbyl Palmitate, Sodium Ascorbyl Palmitate, & Tetrahexyldecyl Ascorbate), Vitamin D3 (Cholecalcipherol), Vitamin Bi-E (Tocopheryl Acetate & Tocopherol/D-Alpha Tocopherol), Alpha Lipoic Acid, Coenzyme Q10, L-Selenothionine, Beta Glucan, Evening Primrose Oil, Sodium Ascorbyl Phosphate, and mixtures thereof present in an amount from about 0.1% to 15% by weight of the composition;
h. PH balancer selected as Potassium Hydroxide or Sodium Hydroxide present in an amount from about 0.01% to 0.15% by weight of the composition;
i. Anti-microbials and Preservatives selected from the group consisting of Sodium Benzoate, Potassium Sorbate, Phenacetin, Methyl Paraben, Propylparaben, Phenoxyethanol, Imidazolidinyl Urea, Tetrasodium EDTA and mixtures thereof and present in an amount from about 0.05% to 10.0% by weight of the composition.

2. The composition according to claim 1, wherein said SD39C alcohol is present in an amount from about 2.0% to 60.0% by weight of the composition.

3. The composition according to claim 1, wherein said water is present in an amount from about 25% to 70.0% by weight of the composition.

4. The composition according to claim 1, wherein said Hepes Linoleate is present in the amount from about 1% to 10% by weight of the composition.

5. The composition according to claim 1, wherein said moisturizer is present as Aloe Vera and Capryylic/Capric triglyceride.

6. The composition according to claim 1, wherein said emulsifier is present as Carbopol 981.

7. The composition according to claim 1, wherein said thickener is a mixture of Sorbitan Oleate and Acrylates/C10-30 Alkyl Acrylate Crosspolymer.

8. The composition according to claim 1, wherein said anti-microbial and preservatives are Tetrasodium EDTA.

9. The composition according to claim 5, further acting as an anti-inflammatory.

10. A method for treating a person afflicted with a chronic inflammatory skin disorder that can develop in the third to sixth decade of life characterized by four distinct clinical stages predominately affecting the central aspect of the face which comprises applying to an affected area of the person's skin in a therapeutically effective amount of a composition consisting of:
a. Water
b. SD39C Alcohol is present in an amount from about 2% to 60% by weight of the composition;
c. Hepes Linoleate is present in an amount from about 0.5% to 20.0% by weight of the composition;
d. Moisturizers selected from the group consisting of sodium pyrollidone carboxylate, glycerin, propylene glycol, sorbitol, Caprylic/Capric Triglyceride, Aloe Vera gel and mixtures thereof present in the amount from about 0.1% to 10% by weight of the composition;
e. Emulsifiers selected from the group PEG-12, Glyceryl Stearate SE, Glyceryl Stearate, Isopropyl Myristate, Aluminum Hydroxide, xanthum gum, Carbopol 981 and mixtures thereof and present in the amount from about 0.1% to 20.0% by weight of the composition;
f. Thickeners selected from the group consisting of Bentonite, Cetyl Alcohol, Carbomer, Sorbitan Oleate, Acrylates/C10-30 Alkyl Acrylate Crosspolymer, and mixtures thereof and present in an amount from about 0.1% to 10.0% by weight of the composition;
g. Anti-microbials and Preservatives selected from the group consisting of Sodium Benzoate, Potassium Sorbate, Phenacetin, Methyl Paraben, Propylparaben, Phenoxyethanol, Imidazolidinyl Urea, Tetrasodium EDTA and mixtures thereof and present in the amount from about 0.05% to 10.0% by weight of the composition.
